(19) United States
(12) Patent Application Publication
(10) Pub. No.: US 2004/0228764 A1
(43) Pub. Date: Nov. 18, 2004

(54) Sampling system

(75) Inventors: Dale Stephens, Victoria (AU); Dirk Kurpershoek, Victoria (AU); Mark Dockrill, Victoria (AU)

Correspondence Address: NIXON & VANDERHYE, PC, 1100 N GLEBE ROAD, 8TH FLOOR, ARLINGTON, VA, 22201-4714, US

(73) Assignee: Ambri Ltd.

(21) Appl. No.: 10/436,450

(22) Filed: May 13, 2003

(51) Int. Cl. G01N 33/48

(52) U.S. Cl. 422/68.1

(57) ABSTRACT

A device and method for sampling a fluid from a container. The device may be used in hospital/clinical diagnostic applications as a point-of-care single-use disposable medical device. The method includes sampling and analysing with the device a fluid sample, such as a biological fluid, from a container.

### FIELD OF THE INVENTION

The present invention relates to a system for sampling a fluid from a container. More particularly the invention relates to the sampling of a biological fluid from a container wherein the contents of the container may be under pressure such as an evacuated collection tube.

### BACKGROUND OF THE INVENTION

Sample analysis is an important part of many industries including food testing, veterinary diagnosis, environmental monitoring and critical care medicine. Sample analysis is an expanding field as the range and number of tests that may be performed on a sample increases daily. Many industries require rapid analysis of samples on site at the point of sampling, without the requirement for complex instrumentation or the need for skilled operators. This is especially so in the critical care medical field.

Sample analysis is predominantly conducted in central laboratories however many manufacturers are increasingly developing point-of-care (POC) test systems to extend the reach of and, in some cases, to replace lab-based systems. This trend towards POC systems is evident as hospital emergency departments, and other critical care units such as operating rooms, trauma and cardiac centres, become the principal centres for the management of moderate and acute illnesses. Medical practice has also evolved such that physicians are more reliant than ever upon laboratory data for the management of their patients.

As the number of patients treated in critical care units has increased, a growing need for rapid diagnosis has developed. However, whilst the proportion of tests conducted in the emergency department has grown, traditional pathology techniques, such as central laboratory testing, have often been inadequate in critical care settings. Furthermore, central laboratory systems are generally complex and expensive and require dedicated, skilled operators who are expected to perform sample preparation, system calibration and basic instrument maintenance.

POC testing provides an inherent advantage over conventional central laboratory testing in, for example, the medical diagnostic field, wherein the diagnostic test may be performed at the patient's bedside and allows for doctors to initiate or adjust the patient's therapy more promptly, i.e. in a matter of minutes rather than hours.

Recent advances in POC analysers, diagnostic reagents and biosensors have offered improved sensitivity, accuracy and precision in laboratory analysis. However problems persist in these systems, primarily in the area of fluid transfer between sample storage or collection units and the testing unit.

Transport of fluid from a storage and handling container to a point of measurement may inherently result in spillage or contamination of the fluid. In the case of blood sampling, one of the obvious associated concerns is the risk of transmission of infectious diseases such as human immunodeficiency virus or hepatitis. This contributes significantly to the risk of sample contamination or infection of the handler, especially in the case of biohazardous fluids, such as blood. These risks are further increased when a container is opened to access the contents or when the contents of a container are under pressure such as a blood sample in an evacuated blood-collection system.

What is needed is a comprehensive fluid sampling system that is capable of eliciting the fluid and transporting it immediately and directly to the measurement and sensing area.

### SUMMARY OF THE INVENTION

In a first aspect the present invention provides a device for use in diagnostic applications, the device comprising a cartridge and a first and second cannula, the first and second cannulas extending from the cartridge; the cartridge comprising a first and a second fluid path, the second fluid path comprising at least one sensing means, the second fluid path being provided with a second port which allows the application of pressure to the second fluid path to move fluid along the second fluid path, the first cannula having a first end connected to the cartridge and a second end having a sharpened tip, the second cannula having a first end connected to the cartridge and a second end having a sharpened tip, the first cannula being in fluid connection with the first fluid path and the second cannula being in fluid connection with the second fluid path, the longitudinal axes of the first and second cannulas being substantially parallel, the first cannula extending from the cartridge beyond the sharpened tip of the second cannula.

In a preferred embodiment the first and second cannulas are integral with the cartridge.

In a preferred embodiment the sharpened tip of each cannula is formed with a sharpened surface that describes a bevelled surface to facilitate penetration of the cannula through resilient closure of a container.

The first cannula and second cannula may be formed from any material that can pierce a pierceable closure of a container and through which a fluid may be drawn. Preferably the cannulas consists of metal, glass or plastic, but it will be appreciated that the cannulas may also consist of combinations of such materials such as plastic coated glass capillary tubes such as those encased in a film of mylar. More preferably the cannulas are stainless steel single bevel non-coring point cannulas. Preferably the cannulas are from 18 to 27 gauge. Most preferably the cannulas are 21 gauge.

In a preferred embodiment the sensing means is a biosensor(s). Preferably the cartridge comprises a plurality of sensing means linked in series, parallel or both series and parallel to provide multiple analysis from a single drawing of fluid from a container. More details regarding biosensors can be found in (International Patent Application Nos. PCT/AU89/00352, PCT/AU90/00025, PCT/AU93/00509, PCT/AU95/00763, PCT/AU96/00482, PCT/AU96/00368, PCT/AU97/00071, PCT/AU97/00014, PCT/AU97/00316, PCT/AU98/00417, PCT/AU98/00423 and PCT/AU98/00424).

In a preferred embodiment the second port is provided on the second fluid path remote from the second cannula. It is preferred that a negative pressure, for example suction, is provided via the second port such that in operation fluid is drawn through the second cannula into and along the second fluid path.

In a preferred embodiment the first fluid path comprises a chamber with a first port such that the chamber is vented to the atmosphere or a buffer system.

Preferably the cartridge has a recessed portion wherein the first cannula and second cannula are housed such that the sharpened tip of each cannula does not project beyond an outer edge of the cartridge.

In another embodiment the cartridge comprises a sensing port by which flow sensing means may sense the flow of fluid along the second fluid path. Preferably the sensing port is an optical sensing port. Preferably the flow of fluid is sensed using an optical sensing device.

In a further preferred embodiment a discharge channel is connected to the second fluid path downstream of the sensing means.

In yet a further preferred embodiment the second fluid path comprises a bubble trap to reduce or eliminate bubbles or other impediments to the flow of fluid through the second fluid path.

In another preferred embodiment the device further comprises a retractable safety shield which selectively encases and provides protection for the sharpened tips of the first cannula and the second cannula.

The cartridge may be manufactured as a single article or comprise an assembly of separate parts such as a housing and a cover, and be formed from a material such as a thermoplastic material. Suitable thermoplastic materials include, but are not limited to, polyethylene, polypropylene, polycarbonate, acrylonitrile/butadiene/styrene (ABS), polyamide, polyacetal, or copolymers of these thermoplastic materials. The cartridge, or cartridge components such as the cartridge housing and cartridge cover may be formed by, for example, die cutting or injection moulding. Preferably the cartridge is disposable.

Preferably the cartridge once formed may be gently heated without substantially affecting the properties of the material and is composed of a material that readily conducts heat to facilitate warming a fluid that has entered the second fluid path when in use.

In a second aspect the present invention provides a method of analysing with a device a fluid sample in a container having a pierceable closure, the device comprising a cartridge and a first and second cannula, the first and second cannulas extending from the cartridge; the cartridge comprising a first and a second fluid path, the second fluid path comprising at least one sensing means, the second fluid path being provided with a second port which allows the application of pressure to the second fluid path to move fluid along the second fluid path, the first cannula having a first end connected to the cartridge and a second end having a sharpened tip, the second cannula having a first end connected to the cartridge and a second end having a sharpened tip, the first cannula being in fluid connection with the first fluid path and the second cannula being in fluid connection with the second fluid path, the longitudinal axes of the first and second cannulas being substantially parallel, the first cannula extending from the cartridge beyond the sharpened tip of the second cannula, the method comprising the steps of:

bringing the container into contact with the device such that the first and second cannulas pierce the pierceable closure;

drawing the fluid sample into the second fluid path via the second cannula; and

displacing the fluid sample in the second fluid path to the sensing means.

Preferably the fluid sample is displaced in the second fluid path to the sensing means by applying a negative pressure, for example suction, via the second port such that in operation fluid is drawn through the second cannula into and along the second fluid path.

In a preferred embodiment the fluid is heated prior to the fluid proceeding to the sensing means. Gentle heating may involve heating the fluid drawn into the second fluid path to a temperature of about 30° C. to about 37° C. Preferably the fluid drawn into the second fluid path is heated to about 33° C. Preferably, the sample in the second fluid path is heated for a predetermined time. Preferably the sample in the second fluid path is heated for about 1 minute to about 5 minutes. More preferably the sample in the second fluid path is heated for about 2 minutes. After heating, the sample may be routed through the sensing means.

In operation, a sample may be collected in to a container by any technique known to those of skill in the art such as a phlebotomist withdrawing a sample of blood using a syringe or catheter. The sample is introduced into a container, such as a Vacutainer® or an evacuated collection tube. The device and container are inserted into a reader instrument and positioned. The Vacutainer® and device of the invention are then brought together, for example by lowering the Vacutainer® onto the first and second cannulas of the device.

The movement of the container towards the cartridge disengages the safety shield, if present, to retract the shield and expose the sharpened tips of the first and second cannula. The Vacutainer® is positioned such that when the Vacutainer® is moved towards the cartridge the first cannula penetrates the pierceable closure of the container and vents the Vacutainer® to the atmosphere. Venting of the Vacutainer® to the atmosphere may result in fluid or gas or a combination passing from the Vacutainer® via the first cannula and into and along the first fluid path and into the chamber. As the container is moved even closer to the cartridge the second cannula penetrates the pierceable closure and is placed in contact with the sample.

Preferably a pressure, such as a negative pressure, is applied through the second fluid path of the cartridge via a port and sample is drawn from the Vacutainer® through the second cannula and into the second fluid path.

It will be appreciated that the sensing means may be connected to a detection system that may measure a parameter of the sensing means such as a chemical or electrical property. The detection system may be a data acquisition system whereby, for example, a sample reaction causes a change in impedance in a membrane in the sensing means and this change in impedance is measured by accurate bridge circuitry and converted to a concentration result, against a programmed algorithm and calibration data. At completion of the test cycle, the device of the invention is released by the reader, and all the interfaces are disconnected.

Preferably the sensing means is a biosensor(s). More preferably the biosensors are linked in series to provide multiple analysis from a single drawing of fluid.

In a preferred embodiment the first fluid path comprises a chamber with a first port such that the chamber is vented to the atmosphere or a buffer system.

While use of the invention is particularly advantageous in the medical environment and will be described in that context, it will be appreciated that the invention may be practiced in any situation where it is desired to perform analysis of fluid samples.

As used herein “container” includes any receptacle that has a resilient closure. The resilient closure may be integral with the container or comprise a cap-type structure that may be removed from the container. Preferably the atmospheric pressure inside the container is greater than the ambient atmospheric pressure outside the container, such as in the case of a Vacutainer®D-type blood collection system. However it must be appreciated that the pressure inside the container may vary depending upon such factors as the length of storage prior to use, and the storage length and conditions after filling with a fluid.

As used herein “fluid” includes any liquid sample that may be analysed. Preferably the fluid is a biological sample and includes biological samples that are able to be suspended in a liquid form from a solid form. More preferably the biological sample is a sample of whole blood, blood preparations such as plasma or EDTA/citrate stabilised samples, urine or saliva. Most preferably the biological sample is whole blood or a blood preparation.

### BRIEF DESCRIPTION OF THE DRAWINGS

These and other features, aspects and advantages of the present invention will become better understood with regard to the following description, appended claims, and accompanying drawings where:

FIG. 1 is a plan view of the device.

FIG. 2 is an exploded perspective view of components of the device.

FIG. 3 is a second exploded perspective view of components of the device.

FIG. 4 is a side view of the bubble trap

### DETAILED DESCRIPTION OF THE PREFERRED EMBODIMENTS

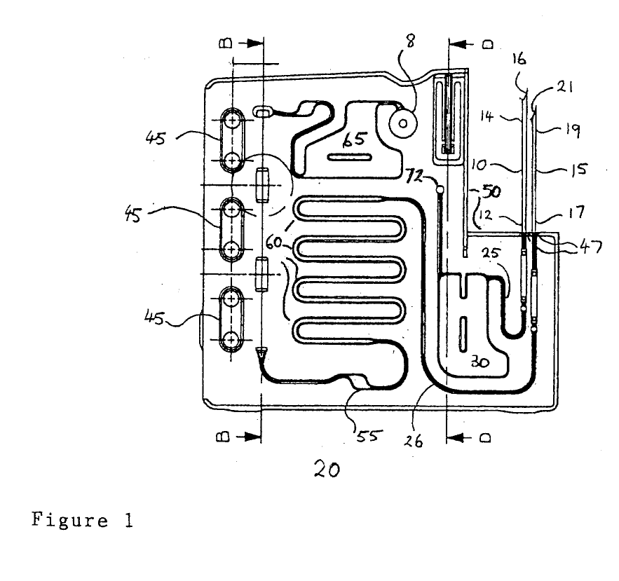

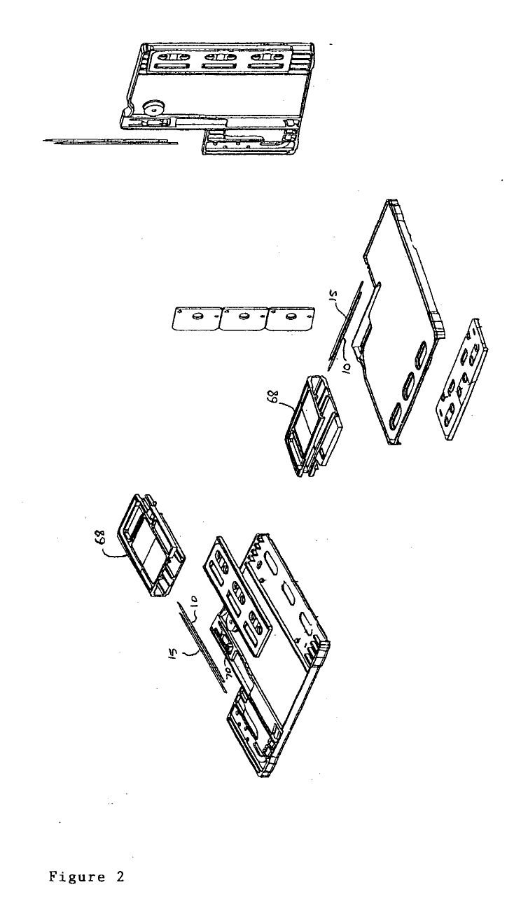

Referring to FIGS. 1-4, a preferred embodiment of the device of the present invention comprises a first cannula 10 connected to a cartridge 20 capable of receiving or drawing a fluid sample and introducing the fluid sample into a first fluid path 25 of a cartridge 20. The cartridge 20 is provided with a second cannula 15 capable of receiving or drawing a fluid sample and introducing the fluid sample into a second fluid path 26 of the cartridge 20. The first fluid path 25 is in fluid connection with a chamber 30 vented to the atmosphere via port 72.

The fluid path 26 is designed to allow an unimpeded flow of sample and to minimise bubble formation.

The first cannula 10 extends from the cartridge 20 and has a sharpened tip 16. The second cannula 15 extends from the cartridge 20 and has a sharpened tip 21. The longitudinal axes of the first 10 and second cannulas 15 are substantially parallel and the first cannula 10 extends from the cartridge 20 beyond the sharpened tip 21 of the second cannula 15.

The sharpened tip 16 and 21 are positioned such that when a container with a pierceable closure is brought into contact with the tips 16 and 21 the sharpened tip 16 of the first cannula 10 pierces the pierceable closure of the container prior to the sharpened tip 21 of the second cannula 15.

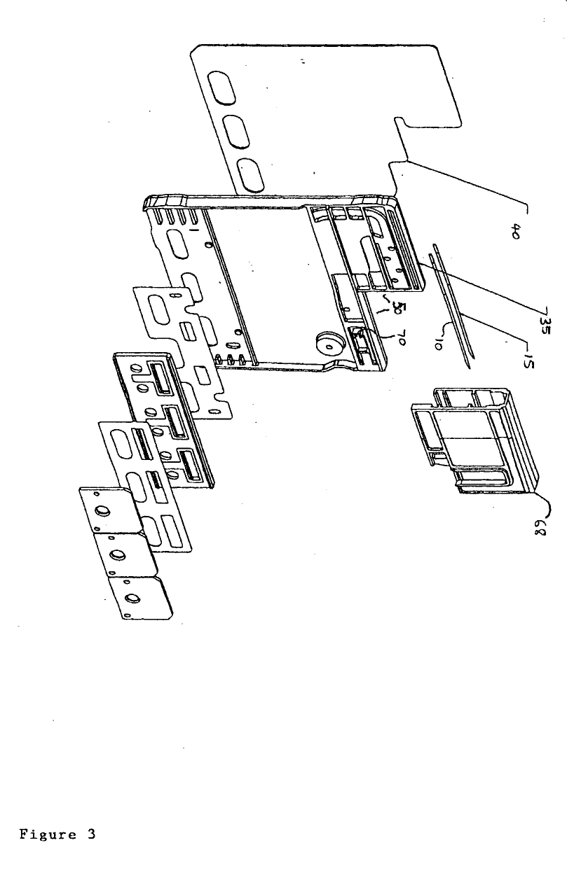

As shown in FIG. 3, the cartridge may be manufactured as a single article or comprise an assembly of separate parts such as a housing 35 and a cover 40.

The first cannula 10 or second cannula 15 may be formed integral with the cartridge 20 or comprise a separate component that may be connected in fluid connection with the cartridge 20. Connection to the cartridge 20 may achieved by insertion of the first end 17 of the first cannula 10 or second cannula 15 into the port 47 of the cartridge 20 wherein a fluid connection is achieved between the first end 17 of the first cannula 10 or second cannula 15 and the port 47.

As shown in FIG. 1, the cartridge 20 has a recessed portion 50 wherein the first cannula 10 and the second cannula 15 are housed such that the sharpened tips 16 and 21 do not project beyond an outer edge of the cartridge 20.

The cartridge 20 includes a sensing port which in FIG. 1 is provided as a transparent window 55 through which the flow of fluid along second fluid path 26 may be sensed. The sensing port provides for a control point whereby in operation a flow sensing means can be used to detect when fluid in the second fluid path 26 has reached the sensing port 55 and then halt the flow of fluid by halting application of pressure, such as a negative pressure force, to the fluid in the second fluid path 26. This provides for the temperature of the sample entering the sensing means 45 to be controlled. The fluid in the serpentine portion 60 of the second fluid path 26 may then be heated by the application of heat from a heating source, to provide a fluid sample at a desired temperature to the sensing means 45. Overflow of sample through the sensing means 45 is collected in discharge channel 65 located downstream of the sensing means 45.

Pressure, such as a negative pressure, may be applied to the second fluid path 26 to draw a fluid through the second cannula 15 and into the second fluid path 26 of the cartridge 20. The pressure may be a negative pressure applied via a port 8 located downstream of the sensing means 45.

As shown in FIG. 3, the device includes a retractable safety shield 68. The retractable safety shield 68 may be disposed about the sharpened tip 16 of the first cannula 10 and second cannula 15 to provide protection of the sharpened tips 16 and 21 of cannulas 10 and 15. A releasible latch 70 is provided which engages the safety shield 68. The releasible latch 70 is disengaged to retract the safety shield 68 and uncover the sharpened tips 16 & 21 of the first cannula 10 and second cannula 15 for operation. Once sampling is complete, the safety shield 68 may be extended and latched with releasable latch 70 to provide a safety shield 68 for the first cannula 10 and second cannula 15. The provision of a safety shield 68 protects the cannulas 10 & 15 from damage and provides safety in transport and use of the device of the present invention.

A port 8 is provided in the second fluid path 26 of the cartridge 20 such that in operation a pressure, such as a negative pressure, can be applied through the port 8 and sample drawn from a container through the second cannula 15 and into the second fluid path 26.

The second fluid path 26 includes a serpentine path 60 in a portion of the second fluid path 26 from the second cannula 15 to the sensing means 45.

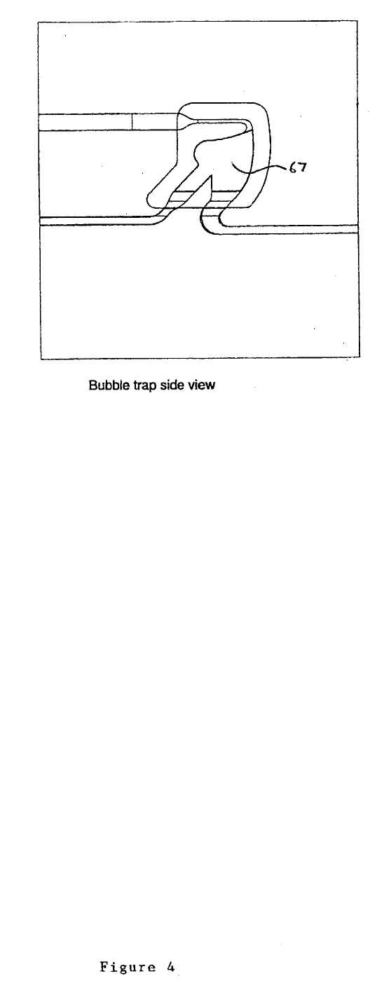

As shown in FIG. 4, a bubble trap 67 may be included in the first fluid path 25 or second fluid path 26 to reduce or eliminate bubbles or other impediments to the flow of fluid through the first fluid path 25 or second fluid path 26.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A device for use in diagnostic applications, the device comprising a cartridge and a first and second cannula, the first and second cannulas extending from the cartridge; the cartridge comprising a first and a second fluid path, the second fluid path comprising at least one sensing means, the second fluid path being provided with a second port which allows the application of pressure to the second fluid path to move fluid along the second fluid path, the first cannula having a first end connected to the cartridge and a second end having a sharpened tip, the second cannula having a first end connected to the cartridge and a second end having a sharpened tip, the first cannula being in fluid connection with the first fluid path and the second cannula being in fluid connection with the second fluid path, the longitudinal axes of the first and second cannulas being substantially parallel, the first cannula extending from the cartridge beyond the sharpened tip of the second cannula.

2. A device according to claim 1 wherein the first and second cannulas are integral with the cartridge.

3. A device according to claim 1 wherein the sharpened tip of each cannula is formed with a sharpened surface that describes a bevelled surface to facilitate penetration of the cannula through resilient closures of a container.

4. A device according to claim 1 wherein the sensing means is a biosensor(s).

5. A device according to claim 1 wherein the cartridge comprises a plurality of sensing means linked in series, parallel or both series and parallel to provide multiple analysis from a single drawing of fluid from a container.

6. A device according to claim 1 wherein the second port is provided on the second fluid path remote from the second cannula.

7. A device according to claim 1 wherein the cartridge has a recessed portion wherein the first cannula and second cannula are housed such that the sharpened tip of each cannula does not project beyond an outer edge of the cartridge.

8. A device according to claim 1 wherein the cartridge comprises a sensing port.

9. A device according to claim 1 wherein a discharge channel is connected to the second fluid path downstream of the sensing means.

10. A device according to claim 1 wherein the second fluid path comprises a bubble trap to reduce or eliminate bubbles or other impediments to flow of fluid through the second fluid path.

11. A device according to claim 1 wherein the device further comprises a retractable safety shield which selectively encases and provides protection for the sharpened tips of the first cannula and the second cannula.

12. A method of analysing with a device a fluid sample in a container having a pierceable closure, the device comprising a cartridge and a first and second cannula, the first and second cannulas extending from the cartridge; the cartridge comprising a first and a second fluid path, the second fluid path comprising at least one sensing means, the second fluid path being provided with a second port which allows the application of pressure to the second fluid path to move fluid along the second fluid path, the first cannula having a first end connected to the cartridge and a second end having a sharpened tip, the second cannula having a first end connected to the cartridge and a second end having a sharpened tip, the first cannula being in fluid connection with the first fluid path and the second cannula being in fluid connection with the second fluid path, the longitudinal axes of the first and second cannulas being substantially parallel, the first cannula extending from the cartridge beyond the sharpened tip of the second cannula, the method comprising the steps of:
bringing the container into contact with the device such that the first and second cannulas pierce the pierceable closure;
drawing the fluid sample into the second fluid path via the second cannula; and
displacing the fluid sample in the second fluid path to the sensing means.

13. A method according to claim 12 wherein the fluid sample is displaced in the second fluid path to the sensing means by applying a negative pressure via the second port such that fluid is drawn through the second cannula into and along the second fluid path.

14. A method according to claim 12 wherein the fluid is heated prior to the fluid proceeding to the sensing means.

15. A method according to claim 12 wherein the first fluid path comprises a chamber with a first port such that the chamber is vented to the atmosphere or a buffer system.

16. A method according to claim 12 wherein the sensing means is a biosensor(s).

17. A method according to claim 12 wherein the sensing means are linked in series to provide multiple analysis from a single drawing of fluid.

18. A method according to claim 12 wherein flow of fluid is sensed using an optical sensing device.
